(19) United States
(12) Patent Application Publication
(10) Pub. No.: US 2021/0267453 A1
(43) Pub. Date: Sep. 2, 2021

(54) SELF-CORRECTING TEMPERATURE AND NOTIFICATION SYSTEM

(71) Applicant: INTERNATIONAL BUSINESS MACHINES CORPORATION, ARMONK, NY (US)

(72) Inventors: Kristina Anne MAULTSBY, Apex, NC (US); Jaime Mae STOCKTON, Groton, MA (US); Stephen GLANCY, Yorktown, VA (US); David XIE, Scarborough (CA)

(21) Appl. No.: 16/802,799

(22) Filed: Feb. 27, 2020

(51) Int. Cl. A61B 5/00; A61F 7/00; G06F 9/54; G08B 25/01; G16H 15/00

(52) U.S. Cl. CPC A61B 5/0008; A61F 7/007; G16H 15/00; G08B 25/016; G06F 9/542

(57) ABSTRACT

A method, computer system, and computer program product for self-correcting temperature and notification are provided. The embodiment may include detecting a user body temperature. The embodiment may also include determining if the detected body temperature is within a pre-configured normal temperature range. The embodiment may further include in response to the detected body temperature is outside the pre-configured threshold temperature range, adjusting the body temperature to the pre-configured normal temperature range. The embodiment may also include notifying a user when the detected body temperature reaches pre-configured dangerous body temperature.

### BACKGROUND

The present invention relates, generally, to the field of computing, and more particularly to body temperature determination and notification systems.

The measurement of body temperature can help detect illness. A high temperature is a fever. Typically, when measuring body temperature, electronic thermometers are mostly used. Electronic thermometers measure a patient's temperature and electronic sensors take a measurement of the temperature. Body temperature represents the balance between heat production and heat loss. Core body temperature may be found in the blood supplying organs such as the brain and those in the abdominal and thoracic cavities. The normal range for core temperature is 36° C.-37.5° C. (96.8° F.-99.5° F.) in clinical practice. An early morning temperature higher than 37.2° C. (99.0° F.) or a late afternoon temperature higher than 37.7° C. (99.86° F.) is normally considered a fever. The high temperature at or above about 40° C. (104° F.) is a life-threatening medical emergency that requires immediate treatment.

### SUMMARY

According to one embodiment, a method, computer system, and computer program product for self-correcting temperature and notification are provided. The embodiment may include detecting a user body temperature. The embodiment may also include determining if the detected body temperature is within a pre-configured normal temperature range. The embodiment may further include in response to the detected body temperature is outside the pre-configured threshold temperature range, adjusting the body temperature to the pre-configured normal temperature range. The embodiment may also include notifying a user when the detected body temperature reaches pre-configured dangerous body temperature.

### BRIEF DESCRIPTION OF THE SEVERAL VIEWS OF THE DRAWINGS

These and other objects, features, and advantages of the present invention will become apparent from the following detailed description of illustrative embodiments thereof, which is to be read in connection with the accompanying drawings. The various features of the drawings are not to scale as the illustrations are for clarity in facilitating one skilled in the art in understanding the invention in conjunction with the detailed description. In the drawings:

FIG. 1 illustrates an exemplary networked computer environment according to at least one embodiment;

FIG. 2 is an operational flowchart illustrating a self-correcting temperature and notification process according to at least one embodiment;

FIG. 3 is a block diagram of internal and external components of computers and servers depicted in FIG. 1 according to at least one embodiment;

FIG. 4 depicts a cloud computing environment according to an embodiment of the present invention; and

FIG. 5 depicts abstraction model layers according to an embodiment of the present invention.

### DETAILED DESCRIPTION

Detailed embodiments of the claimed structures and methods are disclosed herein; however, it can be understood that the disclosed embodiments are merely illustrative of the claimed structures and methods that may be embodied in various forms. This invention may, however, be embodied in many different forms and should not be construed as limited to the exemplary embodiments set forth herein. In the description, details of well-known features and techniques may be omitted to avoid unnecessarily obscuring the presented embodiments.

Embodiments of the present invention relate to the field of computing, and more particularly to body temperature determination and notification systems. The following described exemplary embodiments provide a system, method, and program product to take a user body temperature and self-correct the temperature or send a notification to an appropriate individual to take action. Therefore, the present embodiment has the capacity to improve the technical field of body temperature determination and notification systems by automatically monitoring body temperature and self-correcting the temperature before developing serious issues.

As previously described, a measurement of body temperature can help detect illness. A high temperature is a fever. Typically, when measuring body temperature, electronic thermometers are mostly used. Electronic thermometers measure a patient's temperature and electronic sensors take a measurement of the temperature. Body temperature represents the balance between heat production and heat loss. Core body temperature may be found in the blood supplying organs such as the brain and those in the abdominal and thoracic cavities. The normal range for core temperature is 36° C.-37.5° C. (96.8° F.-99.5° F.) in clinical practice. An early morning temperature higher than 37.2° C. (99.0° F.) or a late afternoon temperature higher than 37.7° C. is normally considered a fever. The high temperature at or above about 40° C. (104° F.) is a life-threatening medical emergency that requires immediate treatment.

Monitoring an individual's body temperature is very important. A user must first recognize that there might be a problem before taking the body temperature of another individual whether the user is a parent with a new baby, an EMT picking up a patient from a 9-1-1 call, or a doctor seeing a patient about a potential illness. Then, a thermometer is used to manually measure the body temperature and the user needs to make a medical decision as a reaction to the temperature reading. Taking other individuals' temperature can be very difficult depending on the situation. For example, an uncooperative child may make temperature measurement very difficult. Similarly, a paramedic may have many difficulties whenever it is necessary to take a patient's temperature in a moving ambulance. Moreover, having opportunities for feedback regarding temperature may let a user determine that something may be wrong prior to the patient having issues. As such, it may be advantageous to, among other things, implement a system that monitors body temperature and sends a notification to an appropriate individual to take necessary action when the body temperature is of concern.

According to one embodiment, the present invention may analyze body temperature to determine whether a user is experiencing abnormal temperature fluctuations. The present invention may also self-correct the body temperature using an electronic or battery-operated device connected to a user mobile device. The present invention may further monitor the measured temperature and, automatically, send a notification to a medical service provider or other appropriate professional or individual when the temperature is at a dangerous level.

The present invention may be a system, a method, and/or a computer program product at any possible technical detail level of integration. The computer program product may include the computer-readable storage medium (or media) having the computer-readable program instructions thereon for causing a processor to carry out aspects of the present invention.

The computer-readable storage medium can be a tangible device that can retain and store instructions for use by an instruction execution device. The computer-readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer-readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer-readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer-readable program instructions described herein can be downloaded to respective computing/processing devices from a computer-readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer-readable program instructions for storage in a computer-readable storage medium within the respective computing/processing device.

Computer-readable program instructions for carrying out operations of the present invention may be assembler instructions, instruction-set-architecture (ISA) instructions, machine instructions, machine-dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer-readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer-readable program instructions by utilizing state information of the computer-readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer-readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions may also be stored in a computer-readable storage medium that can direct a computer, a programmable data processing apparatus, and/or other devices to function in a particular manner, such that the computer-readable storage medium having instructions stored therein comprises an article of manufacture including instructions which implement aspects of the function/act specified in the flowchart and/or block diagram block or blocks.

The computer-readable program instructions may also be loaded onto a computer, other programmable data processing apparatus, or other device to cause a series of operational steps to be performed on the computer, other programmable apparatus or another device to produce a computer-implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The following described exemplary embodiments provide a system, method, and program product for automatically measuring and recording a user body temperature on a regular basis for monitoring and creating a notification to a user when the body temperature indicates that it is in a high or low-temperature range.

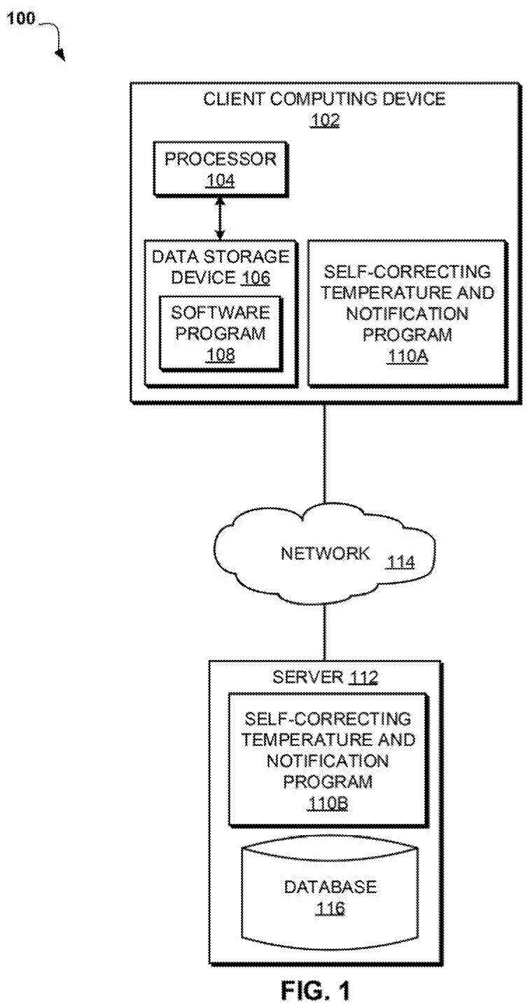

Referring to FIG. 1, an exemplary networked computer environment 100 is depicted according to at least one embodiment. The networked computer environment 100 may include client computing device 102 and a server 112 interconnected via a communication network 114. According to at least one implementation, the networked computer environment 100 may include a plurality of client computing devices 102 and servers 112 of which only one of each is shown for illustrative brevity.

The communication network 114 may include various types of communication networks, such as a wide area network (WAN), local area network (LAN), a telecommunication network, a wireless network, a public switched network and/or a satellite network. The communication network 114 may include connections, such as wire, wireless communication links, or fiber optic cables. It may be appreciated that FIG. 1 provides only an illustration of one implementation and does not imply any limitations with regard to the environments in which different embodiments may be implemented. Many modifications to the depicted environments may be made based on design and implementation requirements.

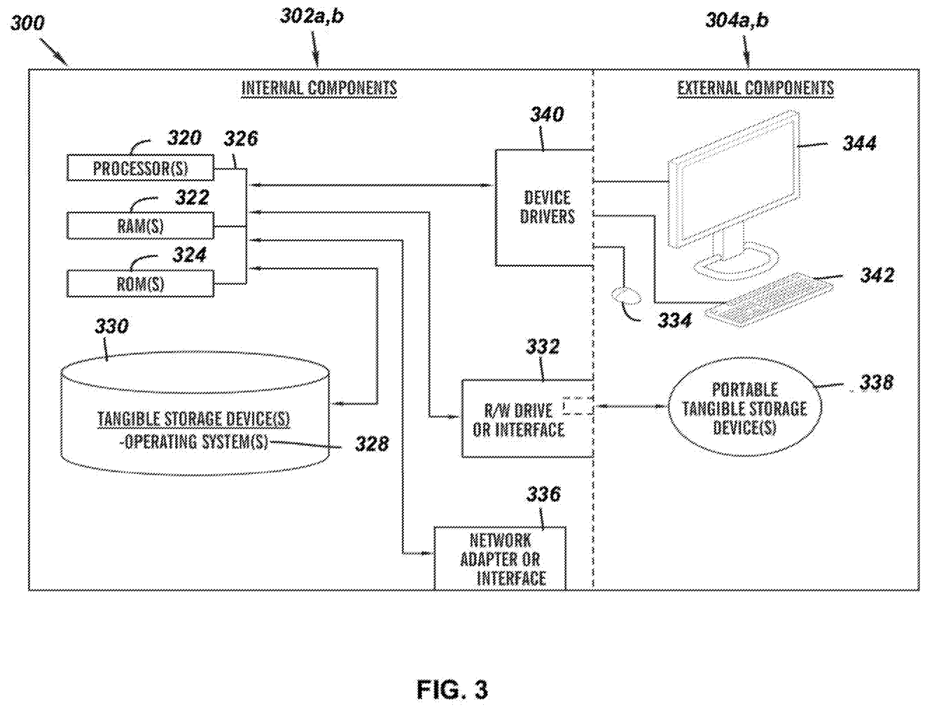

Client computing device 102 may include a processor 104 and a data storage device 106 that is enabled to host and run a software program 108 and a self-correcting temperature and notification program 110A and communicate with the server 112 via the communication network 114, in accordance with one embodiment of the invention. Client computing device 102 may be, for example, a mobile device, a telephone, a personal digital assistant, a netbook, a laptop computer, a tablet computer, a desktop computer, or any type of computing device capable of running a program and accessing a network. As will be discussed with reference to FIG. 3, the client computing device 102 may include internal components 302a and external components 304a, respectively.

The server computer 112 may be a laptop computer, netbook computer, personal computer (PC), a desktop computer, or any programmable electronic device or any network of programmable electronic devices capable of hosting and running a self-correcting temperature and notification program 110B and a database 116 and communicating with the client computing device 102 via the communication network 114, in accordance with embodiments of the invention. As will be discussed with reference to FIG. 3, the server computer 112 may include internal components 302b and external components 304b, respectively. The server 112 may also operate in a cloud computing service model, such as Software as a Service (SaaS), Platform as a Service (PaaS), or Infrastructure as a Service (IaaS). The server 112 may also be located in a cloud computing deployment model, such as a private cloud, community cloud, public cloud, or hybrid cloud.

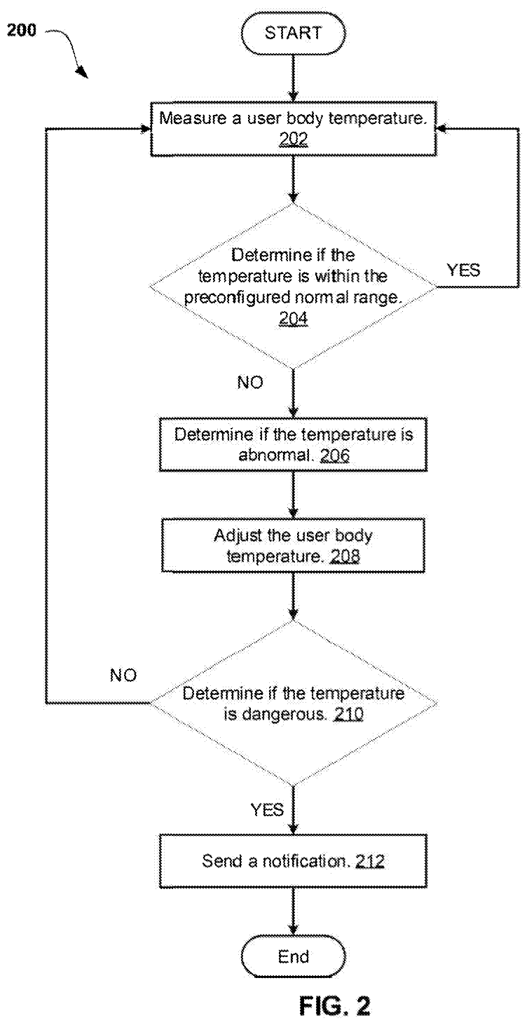

According to the present embodiment, the self-correcting temperature and notification program 110A, 110B may be a program capable of measuring and recording a user's body temperature and determining whether special attention is needed via a user mobile device. The self-correcting temperature and notification process are explained in further detail below with respect to FIG. 2.

Referring to FIG. 2, an operational flowchart illustrating a self-correcting temperature and notification process 200 is depicted according to at least one embodiment. At 202, the self-correcting temperature and notification program 110A, 110B measures a user body temperature. According to one embodiment, the self-correcting temperature and notification program 110A, 110B may utilize an electronic or battery-operated device attached to various objects, such as a crib, a bed, a stretcher, a chair or a table, to automatically measure a user's body temperature. For example, electronic or battery-operated wiring sewed into a mattress cover, sheet or blanket may be used to measure an infant or a patient's body temperature at preconfigured times. In one other embodiment, the self-correcting temperature and notification program 110A, 110B may utilize any temperature sensing device. For example, a headband worn by a patient that includes a temperature sensor may be utilized to measure the patient's body temperature. Mobile or computer applications may be used to control and monitor such electronic devices.

At 204, the self-correcting temperature and notification program 110A, 110B determines if the measured temperature is within the preconfigured normal range. According to one embodiment, the self-correcting temperature and notification program 110A, 110B may compare the above-measured temperature to a medically-accepted normal temperature range, which is usually in the range of 36.5° C. (97.7° F.) to 37.5° C. (99.5° F.). In at last one other embodiment, the self-correcting temperature and notification program 110A, 110B may search and retrieve temperature-related data from various databases to collect a more narrowly targeted body temperature range for specific age groups, body size, weights, heights, family history and ethnicity, if appropriate. For example, an infant with the previous medical history of hyperthermia or heat intolerance may require closer attention to a slight change in the body temperature, and thus, the self-correcting temperature and notification program 110A, 110B may apply a slightly modified normal temperature range when monitoring the temperature of the infant in this example. If the measured temperature is within the normal temperature range (step 204, “Yes” branch), then the self-correcting temperature and notification program 110A, 110B may return to step 202 to measure the user body temperature again. In yet another embodiment, the self-correcting temperature and notification program 110A, 110B may wait a preconfigured period of time before re-measuring the user's body temperature. For example, a different individual may have a different temperature tolerance, such that the self-correcting temperature and notification program 110A, 110B may apply different wait times to different users. If the measured temperature is not within the normal temperature range (step 204, “No” branch), then the self-correcting temperature and notification program 110A, 110B may proceed to step 206 to determine whether the measured temperature is abnormal.

At 206, the self-correcting temperature and notification program 110A, 110B determines if the temperature is abnormal. According to one embodiment, the self-correcting temperature and notification program 110A, 110B may determine that the measured body temperature is too high based on the pre-configured temperature range or medically accepted range for a specific user type. For example, the self-correcting temperature and notification program 110A, 110B may configure that the high temperature is any temperature above 37.5° C. (99.5° F.) and blow 38.3° C. (101.0° F.) and the measured temperature is at 37.8° C. (100.0° F.), then the self-correcting temperature and notification program 110A, 110B may prompt a cooling device to cool down the body temperature. For example, a cooling device may be a cooling pad with a temperature sensor attached to a blanket or a mattress that may help lower a patient's body temperature.

At 208, the self-correcting temperature and notification program 110A, 110B adjusts the user body temperature. According to one embodiment, the self-correcting temperature and notification program 110A, 110B may direct an associated device to provide warmth to a user thereby increasing the user body temperature when the self-correcting temperature and notification program 110A, 110B determines that the measured body temperature is below a preconfigured temperature. The self-correcting temperature and notification program 110A, 110B may interact with a heating device, such as electronic or battery-operated wiring sewn into a stretch cover, mattress cover, sheet or blanket which may connect to a user mobile application or desktop application. In at least one other embodiment, a user may manually control the above electronic or battery-operated wiring device to adjust the temperature. In yet another embodiment, the self-correcting temperature and notification program 110A, 110B may cool down the measured body temperature using the above electronic or battery-operated wiring systems. For example, if an infant's body temperature is measured and it indicates that the temperature needs to be lowered, then a pad with the above wiring systems may cool down the pad and the self-correcting temperature and notification program 110A, 110B may monitor the infant's body temperature to check if the temperature drops to the desired temperature or preconfigured normal temperature range. In at least one other embodiment, the self-correcting temperature and notification program 110A, 110B may send a notification regarding the infant's body temperature in an increased frequency as the self-correcting temperature and notification program 110A, 110B has once triggered a temperature adjustment operation.

At 210, the self-correcting temperature and notification program 110A, 110B determines if the temperature is dangerous. Dangerous temperatures may be high-grade fevers that range from over 40.0° C. (104° F.)-41.7° C. (107° F.) or higher. According to one embodiment, the self-correcting temperature and notification program 110A, 110B may determine that the measured temperature is at a dangerous level when the above temperature adjustment attempts do not change the initially measured body temperature or fails to adjust it to the preconfigured normal range. In at least one other embodiment, the self-correcting temperature and notification program 110A, 110B may bypass the above temperature adjustment step when an initially measured body temperature is abnormal such that the temperature is already at the dangerous level. In one embodiment, any temperature above or below the medically-accepted normal temperature range (36.5° C. (97.7° F.) to 37.5° C. (99.5° F.)) may be configured to be dangerous. In yet another embodiment, appropriate personnel, such as a medical service provider, a parent or a guardian, may manually set the dangerous temperature level based on a user's medical history.

At 212, the self-correcting temperature and notification program 110A, 110B sends a notification to an appropriate person. According to one embodiment, the self-correcting temperature and notification program 110A, 110B may utilize a wiring device that may detect body temperature and send the recording to healthcare professionals alerting the temperature fluctuations. The self-correcting temperature and notification program 110A, 110B may store emergency contacts and when the primary contact fails to respond to any push notification regarding temperature fluctuations in a certain amount of time, a secondary contact may be notified. In one embodiment, the self-correcting temperature and notification program 110A, 110B may notify emergency responders, such as dialing 9-1-1, if no emergency contacts may be reached in a preconfigured amount of time. In yet another embodiment, the self-correcting temperature and notification program 110A, 110B may connect to an electronic health record system when the measured temperature is considered dangerous and notify appropriate healthcare professionals to take appropriate actions.

It may be appreciated that FIGS. 2 provides only an illustration of one implementation and does not imply any limitations with regard to how different embodiments may be implemented. Many modifications to the depicted environments may be made based on design and implementation requirements. For example, in at least one embodiment, the self-correcting temperature and notification program 110A, 110B may send notifications to a user's primary contacts to take specific, necessary actions while waiting for an ambulance or a healthcare professional's attention. For example, such actions may be providing a fever reducer or taking off a patient's clothing.

FIG. 3 is a block diagram of internal and external components of the client computing device 102 and the server 112 depicted in FIG. 1 in accordance with an embodiment of the present invention. It should be appreciated that FIG. 3 provides only an illustration of one implementation and does not imply any limitations with regard to the environments in which different embodiments may be implemented. Many modifications to the depicted environments may be made based on design and implementation requirements.

The data processing system 302, 304 is representative of any electronic device capable of executing machine-readable program instructions. The data processing system 302, 304 may be representative of a smartphone, a computer system, PDA, or other electronic devices. Examples of computing systems, environments, and/or configurations that may represented by the data processing system 302, 304 include, but are not limited to, personal computer systems, server computer systems, thin clients, thick clients, hand-held or laptop devices, multiprocessor systems, microprocessor-based systems, network PCs, minicomputer systems, and distributed cloud computing environments that include any of the above systems or devices.

The client computing device 102 and the server 112 may include respective sets of internal components 302a,b and external components 304a,b illustrated in FIG. 3. Each of the sets of internal components 302 include one or more processors 320, one or more computer-readable RAMs 322, and one or more computer-readable ROMs 324 on one or more buses 326, and one or more operating systems 328 and one or more computer-readable tangible storage devices 330. The one or more operating systems 328, the software program 108 and the self-correcting temperature and notification program 110A in the client computing device 102 and the self-correcting temperature and notification program 110B in the server 112 are stored on one or more of the respective computer-readable tangible storage devices 330 for execution by one or more of the respective processors 320 via one or more of the respective RAMs 322 (which typically include cache memory). In the embodiment illustrated in FIG. 3, each of the computer-readable tangible storage devices 330 is a magnetic disk storage device of an internal hard drive. Alternatively, each of the computer-readable tangible storage devices 330 is a semiconductor storage device such as ROM 324, EPROM, flash memory or any other computer-readable tangible storage device that can store a computer program and digital information.

Each set of internal components 302a,b also includes an R/W drive or interface 332 to read from and write to one or more portable computer-readable tangible storage devices 338 such as a CD-ROM, DVD, memory stick, magnetic tape, magnetic disk, optical disk or semiconductor storage device. A software program, such as a self-correcting temperature and notification program 110A, 110B can be stored on one or more of the respective portable computer-readable tangible storage devices 338, read via the respective R/W drive or interface 332 and loaded into the respective hard drive 330.

Each set of internal components 302a,b also includes network adapters or interfaces 336 such as a TCP/IP adapter cards, wireless Wi-Fi interface cards, or 3G or 4G wireless interface cards or other wired or wireless communication links. The software program 108 and the self-correcting temperature and notification program 110A in the client computing device 102 and the self-correcting temperature and notification program 110B in the server 112 can be downloaded to the client computing device 102 and the server 112 from an external computer via a network (for example, the Internet, a local area network or other, wide area network) and respective network adapters or interfaces 336. From the network adapters or interfaces 336, the software program 108 and the self-correcting temperature and notification program 110A in the client computing device 102 and the self-correcting temperature and notification program 110B in the server 112 are loaded into the respective hard drive 330. The network may comprise copper wires, optical fibers, wireless transmission, routers, firewalls, switches, gateway computers and/or edge servers.

Each of the sets of external components 304a,b can include a computer display monitor 344, a keyboard 342, and a computer mouse 334. External components 304a,b can also include touch screens, virtual keyboards, touch pads, pointing devices, and other human interface devices. Each of the sets of internal components 302a,b also includes device drivers 340 to interface to computer display monitor 344, keyboard 342, and computer mouse 334. The device drivers 340, R/W drive or interface 332, and network adapter or interface 336 comprise hardware and software (stored in storage device 330 and/or ROM 324).

It is understood in advance that although this disclosure includes a detailed description on cloud computing, implementation of the teachings recited herein is not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g. networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is a service oriented with a focus on statelessness, low coupling, modularity, and semantic interoperability. At the heart of cloud computing is an infrastructure comprising a network of interconnected nodes.

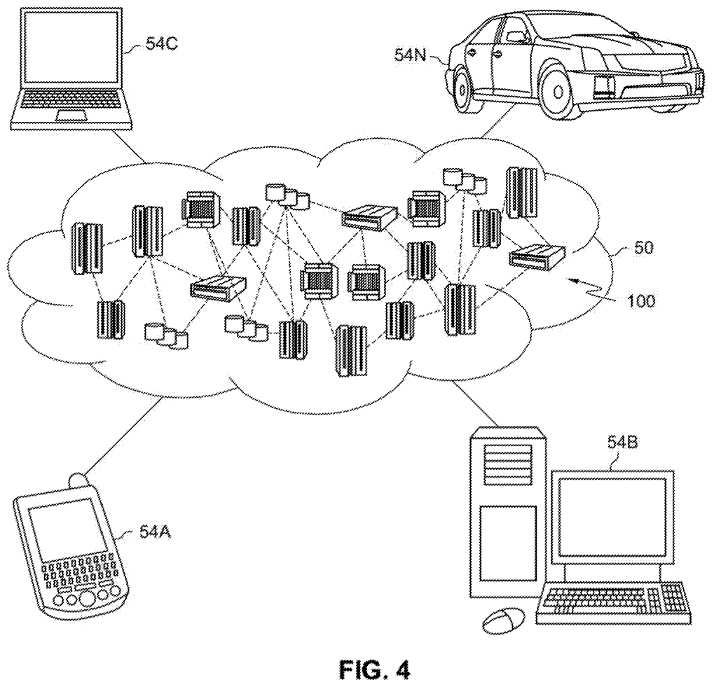

Referring now to FIG. 4, illustrative cloud computing environment 50 is depicted. As shown, cloud computing environment 50 comprises one or more cloud computing nodes 100 with which local computing devices used by cloud consumers, such as, for example, personal digital assistant (PDA) or cellular telephone 54A, desktop computer 54B, laptop computer 54C, and/or automobile computer system 54N may communicate. Nodes 100 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 4 are intended to be illustrative only and that computing nodes 100 and cloud computing environment 50 can communicate with any type of computerized device over any type of network and/or network addressable connection (e.g., using a web browser).

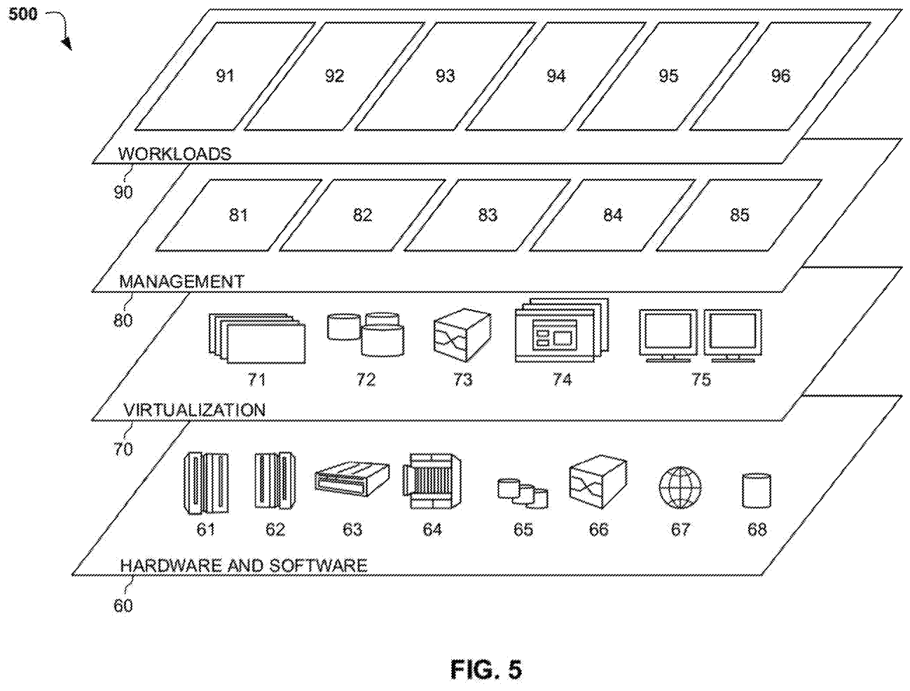

Referring now to FIG. 5, a set of functional abstraction layers 500 provided by cloud computing environment 50 is shown. It should be understood in advance that the components, layers, and functions shown in FIG. 5 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and self-correcting temperature and notification 96. Self-correcting temperature and notification 96 relate to monitoring a user body temperature and sending notifications to appropriate personnel regarding temperature fluctuations.

The descriptions of the various embodiments of the present invention have been presented for purposes of illustration but are not intended to be exhaustive or limited to the embodiments disclosed. Many modifications and variations will be apparent to those of ordinary skill in the art without departing from the scope of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A processor-implemented method for self-correcting temperature notification, the method comprising:
detecting a user body temperature;
determining if the detected body temperature is within a pre-configured normal temperature range; and
in response to the detected body temperature being outside the pre-configured normal temperature range, performing temperature regulation assistance using associated temperature regulation devices to adjust the user body temperature to the pre-configured normal temperature range.

2. The method of claim 1, further comprising:
notifying a user when the detected body temperature is within a pre-configured dangerous body temperature; and
sending a notification to the user via a mobile application or a desktop application.

3. The method of claim 1, further comprising:
storing primary contacts of the user in a database; and
sending a notification to each primary contact when the detected body temperature reaches the pre-configured dangerous body temperature.

4. The method of claim 1, further comprising:
utilizing an electronic system attached to a bed, a chair, a stretcher or a wearable to adjust the body temperature.

5. The method of claim 3, further comprising:
notifying an emergency center when each primary contact does not respond within a preconfigured time.

6. The method of claim 1, further comprising:
recommending a necessary action when the detected body temperature is outside the pre-configured normal temperature range, wherein the necessary action is any action that may help increase or decrease the body temperature.

7. The method of claim 1, further comprising:
storing temperature measurements in a database connected to a user electronic health record system.

8. A computer system for self-correcting temperature notification, the computer system comprising:
one or more processors, one or more computer-readable memories, one or more computer-readable tangible storage media, and program instructions stored on at least one of the one or more tangible storage media for execution by at least one of the one or more processors via at least one of the one or more memories, wherein the computer system is capable of performing a method comprising:
detecting a user body temperature;
determining if the detected body temperature is within a pre-configured normal temperature range;
in response to the detected body temperature is outside the pre-configured normal temperature range, adjusting the user body temperature to the pre-configured normal temperature range; and
notifying a user when the detected body temperature reaches pre-configured dangerous body temperature.

9. The computer system of claim 8, further comprising:
sending a notification to the user via a mobile application or a desktop application.

10. The computer system of claim 8, further comprising:
storing primary contacts of the user in a database; and
sending a notification to the primary contacts when the detected body temperature reaches the pre-configured dangerous body temperature.

11. The computer system of claim 8, further comprising:
utilizing an electronic or battery-operated wiring system attached to a bed, a chair, a stretcher or a wearable to adjust the body temperature.

12. The computer system of claim 10, further comprising:
notifying an emergency center when the primary contacts do not respond within a preconfigured time.

13. The computer system of claim 8, further comprising:
recommending a necessary action when the detected body temperature is outside the pre-configured normal temperature range.

14. The computer system of claim 8, further comprising:
storing temperature measurements in a database connected to a user electronic health record system.

15. A computer program product for self-correcting temperature notification, the computer program product comprising:
one or more computer-readable tangible storage media and program instructions stored on at least one of the one or more tangible storage media, the program instructions executable by a processor of a computer to perform a method, the method comprising:
detecting a user body temperature;
determining if the detected body temperature is within a pre-configured normal temperature range;
in response to the detected body temperature is outside the pre-configured normal temperature range, adjusting the user body temperature to the pre-configured normal temperature range; and
notifying a user when the detected body temperature reaches pre-configured dangerous body temperature.

16. The computer program product of claim 15, further comprising:
sending a notification to the user via a mobile application or a desktop application.

17. The computer program product of claim 15, further comprising:
storing primary contacts of the user in a database; and
sending a notification to the primary contacts when the detected body temperature reaches the pre-configured dangerous body temperature.

18. The computer program product of claim 15, further comprising:
utilizing an electronic or battery-operated wiring system attached to a bed, a chair, a stretcher or a wearable to adjust the body temperature.

19. The computer program product of claim 17, further comprising:
notifying an emergency center when the primary contacts do not respond within a preconfigured time.

20. The computer program product of claim 15, further comprising:
storing temperature measurements in a database connected to a user electronic health record system.
